(19) United States
(12) Patent Application Publication
(10) Pub. No.: US 2019/0122767 A1
(43) Pub. Date: Apr. 25, 2019

(54) SYSTEM, DEVICES, AND METHODS FOR CODING AND DECODING MOTION ACTIVITY AND FOR DETECTING CHANGE IN SUCH

(71) Applicant: Arizona Board of Regents on Behalf of the University of Arizona, Tucson, AZ (US)

(72) Inventor: Marvin J. Slepian, Tucson, AZ (US)

(21) Appl. No.: 16/309,757

(22) PCT Filed: Jun. 16, 2017
(86) PCT No.: PCT/US2017/037949

§ 371 Date: Dec. 13, 2018

Related U.S. Application Data
(60) Provisional application No. 62/351,264, filed on Jun. 16, 2016.

(51) Int. Cl. G16H 50/20; G06F 21/60; G16H 40/63; G16H 20/30

(52) U.S. Cl. CPC G16H 50/20; G06F 21/602; G06F 16/2457; G16H 20/30; G16H 40/63

(57) ABSTRACT

Systems and methods detect changes in ability of an individual to perform an activity. The system includes a processor, a memory, a generic signature database having at least one signature that corresponds to the activity, a decryptor that identifies a current activity of the individual, a normal activity pattern database that includes a pattern of the activity previously determined for the individual, and an activity analyzer configured to compare performance of the current activity to previous performances of the activity to identify change in the performance, and to generate an alert indicating the change when the change is identified. Sensor data indicative of movement of the individual is processed to identify the current activity and performance of the current activity is compared to previous performances of the activity to identify change and an alert is generated to indicate the change in the ability of the individual to perform the activity.

### RELATED APPLICATIONS

This application claims priority to U.S. Patent Application Ser. No. 62/351,264, titled “System, Devices, and Methods for Coding and Decoding Motion Activity and for Detecting Change in Such”, filed Jun. 16, 2016, and incorporated herein by reference.

### BACKGROUND

Activity of an organism, including man, has associated movement(s). Conventionally these movements are described qualitatively (e.g. a person travelled far), semi-quantitatively (e.g. the individual walked one block), or measured to provide quantitative movement (e.g. the individual walked fifty feet).

Each activity, movement, and motion has multiple components to that act. These activities may include three or four-dimensional information, such as displacement and rotation in the x, y and z plane (e.g., or six degrees of freedom), time information (i.e., the fourth-dimension), that gives one or more of rate, velocity and acceleration information, and so on.

### SUMMARY

Although devices (e.g., “Fit bit, “Up Band”, and smart phone) visually or digitally capture motion, such as in the number of steps for example, no system or method exists to systematically describe, define, capture and digitally code or encrypt motions, activities or other motion related processes (e.g., handwriting, typing and other activities of daily living such as opening a pill bottle, drinking from a cup, buttoning a shirt).

Further, even if a motion activity is somehow codified or encrypted in some type of signal means or form, no system exists to systematically de-encrypt or de-codify or otherwise interpret and extract the motion information and translate it for use.

In the embodiments described herein, movement may be determined by one of three ways: implantable sensors that measure movement from within the body, wearable sensors that attach to the body to determine movement, and off the body sensing, where body movements is determined using external apparatus, such as in a wired room that utilizes one or more of machines and cameras to detect body movement. The detected movement may be quantitative and continuous directly outputted, derived or processed signals may be utilized to provide a digital (via either directly digital or analog to digitally converted) signal of the motion. These signals for a given motion then become the codification, encryption and specific or unique “signature” of a given motion or of the activity, activity component or effect represented by the motion.

Conversely, as more and more motions, activities or activity components are captured and codified or encrypted an effective code, lexicon and language of signatures may be established. As such, this language, code or lexicon may be utilized to decipher interpret, de-encrypt or otherwise translate a given motion signal or signature. In the embodiments hereof, this process may either be done manually, semi-manually or via an automated system.

In the embodiments hereof, the activity or activity component of an organism may be fully revealed, without visual or any other direct sensory input directly from the motion signature.

Additionally, as health of an individual changes, the ability of that individual to perform certain actions, motions and activities also changes. Such changes may be gradual and thereby unnoticed by the individual and/or other monitoring the individual. Particularly, where the individual lives at home alone, the gradual deterioration of the individual's health may go unnoticed until a significant event occurs.

In the embodiments disclosed herein, the captured and interpreted motion signature(s) may be analyzed over time or over differing circumstances or contexts. As such, alterations in the motion signature over time or context may reveal a decline in function, state of health or other endogenous or exogenous affecting condition being at work. Hence the motion signature may provide insight as to both the local and general status of function, health, mental or physical intactness or otherwise form and functionality of an individual.

In an embodiment, a system detects changes in ability of an individual to perform an activity. The system includes a processor, a memory, a generic signature database stored within the memory and having at least one signature that corresponds to the activity, a decryptor that identifies a current activity being performed by the individual by matching one or more signatures, determined from sensor data received from sensing devices attached to the individual, to the at least one signature within the generic signature database, a normal activity pattern database stored within the memory and that includes a pattern of the activity previously determined for the individual, and an activity analyzer configured to compare performance of the current activity to performance of the previous activity to identify change in the performance, and to generate an alert indicating the change when the change is identified.

In another embodiment, a method detects changes in ability of an individual to perform an activity. Sensor data indicative of movement of the individual is processed to identify a current activity of the individual. Performance of the current activity is compared to previous performances of the activity by the individual to identify change in the ability of the individual to perform the activity. An alert is generated to indicate the change in the ability of the individual to perform the activity.

### BRIEF DESCRIPTION OF THE FIGURES

FIG. 1A shows one exemplary system for capturing, coding and/or encrypting, analyzing, storing and refining (Via machine language feedback to refine) motion signatures to create a library database, in an embodiment.

FIG. 1B is a flowchart illustrating one exemplary method 100 for capturing, coding and/or encrypting, analyzing, storing and refining (Via machine language feedback to refine) motion signatures to create a library database, in an embodiment.

FIGS. 2 and 3 show a right side view and a front view of an individual showing exemplary positioning of the sensing devices of FIG. 1, in an embodiment.

FIG. 4 shows one exemplary system for capturing and decrypting sensor data by comparing a signal to the library database thereby interpreting without any other input (e.g. visual) what the motion or motion component is

FIG. 5 shows one exemplary system for detecting change in ability of an individual based upon identified actions, in an embodiment.

FIG. 6 is a flowchart illustrating one example method for detecting change in ability of an individual based upon identified actions, in an embodiment.

FIGS. 7 and 8 show a scenario of an individual with a sensing device attached to a hand, in an embodiment.

FIGS. 9, 10, and 11 shows graphs of exemplary sensor data collected from the sensing device of FIGS. 7 and 8, as the individual writes letters of the alphabet, in an embodiment.

### DETAILED DESCRIPTION OF THE EMBODIMENTS

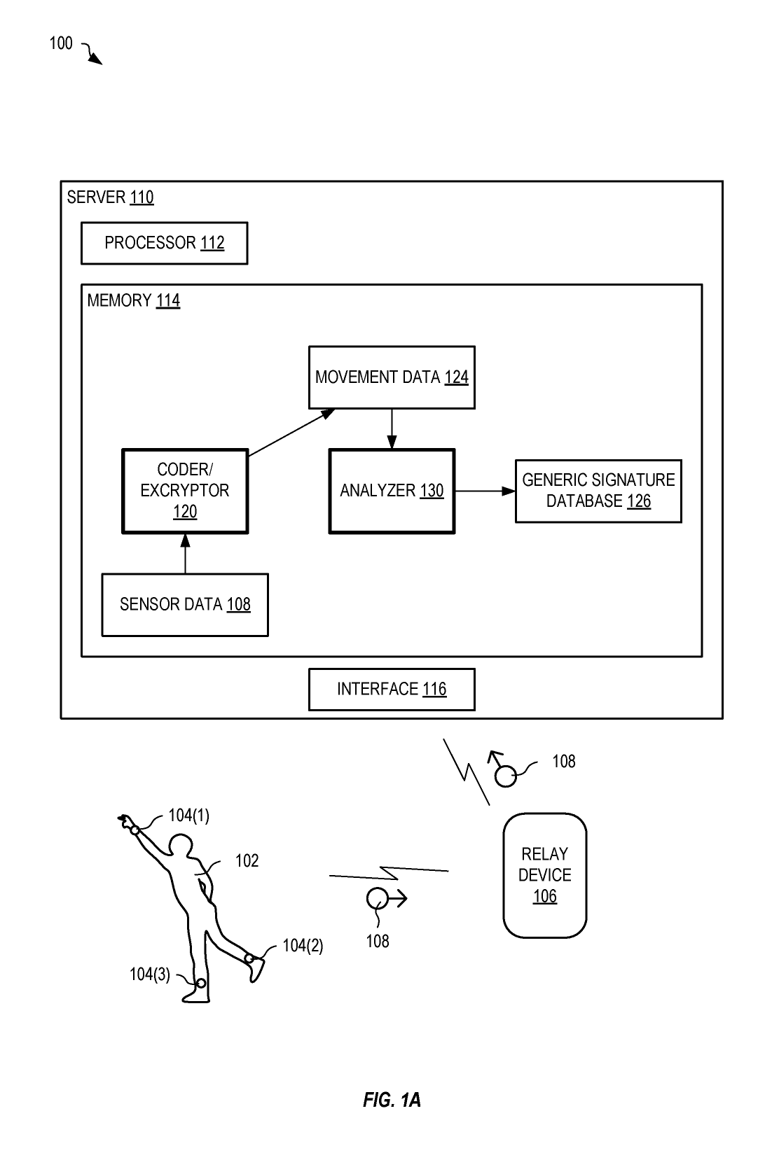

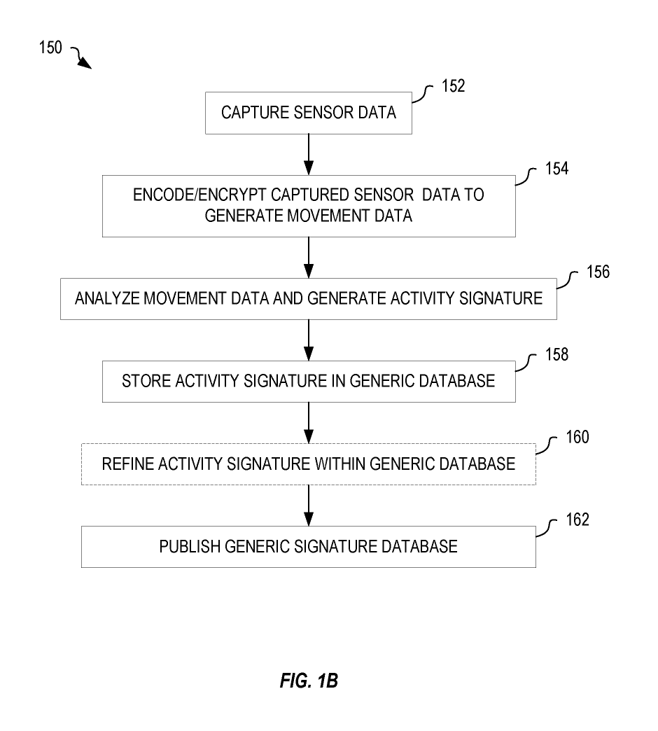

FIG. 1A shows one exemplary system for capturing, coding and/or encrypting, analyzing, storing and refining (via machine language feedback to refine) motion signatures to create a library database. System 100 may be used for detecting change in ability of an individual 102 based upon identified actions. System 100 includes a server 110, implemented as a computer that includes at least one processor 112 and memory 114. Server 110 may be implemented “in the cloud” and be accessible via the Internet, as known in the art. An individual 102 has at least one sensing device 104 that is configured to monitor motion of individual 102. As shown in FIG. 1, individual 102 has a first sensing device 104(1) positioned at a wrist of one arm, a second sensing device 104(2) positioned at a right ankle, and a third sensing device 104(3) positioned at a left ankle. Individual 102 may have more or fewer sensing devices 104 without departing from the scope hereof. Sensing device 104 may include one or more sensors selected from the group including strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, vibration sensors, and so on. Engineering has produced a plethora of sensors for all types of measurement and miniaturization of sensors and development of flexible circuitry allows sensing devices 104 to be implanted within the body of individual 102, affixed to the body of individual 102, and incorporated into clothing of individual 102 such that continuous collection of movement information is possible without disruption of activity of individual 102. See for example, www.MC10inc.com (e.g. the “Biostamp”) for examples of sensing devices. The embodiments hereof may use any type of sensor and any format as best suited to the measurements needed.

For example, sensing device 104 may be implemented as a wearable thin appliqué stretchable electronic device that contains accelerometers and gyroscopes that provide three dimensional coordinates as well as overall 3-D spatial values indicative of motion of individual 102. In one example of operation, each sensing device 104 senses X-Y, Y-Z and X-Z directionality of motion, dimension data, velocity data and acceleration data for each plane may be measured and/or derived through integration. Each sensing device 104 sends sensor data to server 110 for analysis. As shown in FIG. 1, individual 102 may utilized a relay device 106 (e.g., a smart phone, or other such similar communication capable device) to receive sensor data 108 from each sensing device 104, and to relay that sensor data 108 to server 110. For example, where relay device 106 is a smart phone, relay device 106 may communicate with server 110 via the internet and other computer networks, as known in the art. As individual 102 performs an activity 109, sensing devices 104 collect sensors data 108, which is sent to server 110, optionally via a relay device 106.

Within server 110, a coder/encryptor 120 processes captured sensor data 108 to determine movement data 124. An analyzer 130 analyzes movement data 124 to generate generic signature database 126.

Although FIG. 1 shows a single individual 102, server 110 operates to capture, code, and analyze sensor data 108 repeatedly from the same individual and also from other individuals to build a robust generic signature database 126.

System 100 is a “learning system”, which updates generic signature database 126 with movement signatures as new activities are learned. For example, where sensor data 108 does not correlate to existing signatures within generic signature database 126, server 102 may utilize additional information (e.g., from an external device or administrator) to identify the activity defined within sensor data 108, and then update generic signature database 126 to include signatures, or sequences thereof, for identifying that activity. In one embodiment, generic signature database 126 is shared between a plurality of servers 110, each collecting and processing sensor data 108 from many individuals, whereby system 100 learns new activities very quickly.

Activities identified by coder/encryptor 120 may include writing of the alphabet (in both cursive as well as print), opening of ajar, lifting and sipping of a coffee cup, cutting meat, buttoning a shirt, and so on. As described above, system 100 may learn any common conventional activity, such that it is defined within generic signature database 126 and used to determine current activity 124 based upon sensor data 108.

FIG. 1B is a flowchart illustrating one exemplary method 100 for capturing, coding and/or encrypting, analyzing, storing and refining (Via machine language feedback to refine) motion signatures to create a library database.

In step 152, method 150 captures movement data. In one example of step 152, sensing device(s) 104 sense movement of individual 102 and send sensor data 108 to interface 116 of server 110. In step 154, method 150 encodes/encrypts captured movement data. In one example of step 154, coder/entryptor 120 processes sensor data 108 to generate movement data 124. In step 156, method 150 analyzes encoded movement data to generate an activity signature. In one example of step 156, analyzer 130 processes movement data 124 to generate a movement signature. In step 158, method 150 stores the activity signature within a generic database. In one example of step 158, analyzer 130 stores the generated movement signature within generic signature database 126. Step 160 is optional. If included, in step 160, method 150 refines the activity signature within the generic database. In one example of step 160, server 110 may repeatedly receive sensor data 108 for similar activities and refines the associated generic signature within generic signature database 126.

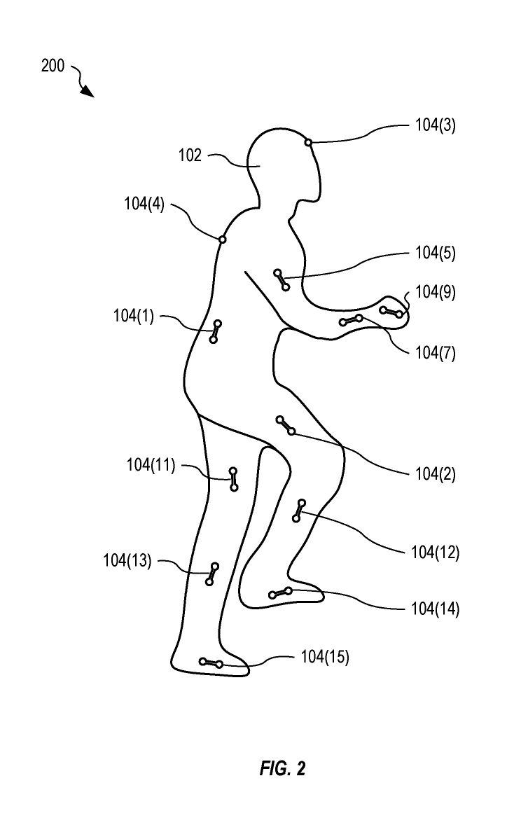

FIG. 2 is a right side view 200 of individual 102 and FIG. 3 is a front view 300 of individual 102, each view 200, 300 illustrating example positioning of sensing devices 104 on individual 102. FIGS. 2 and 3 are best viewed together with the following description.

Sensing device 104(1) is positioned at the lower-back area of individual 102 and sensing device 104(2) is positioned at a right-thigh area of individual 102. Sensing device 104(3) is positioned on the head of individual 102 and measures head movement. In one embodiment, sensing device 104(3) is configured with a hat worn by individual 102. Sensing device 104(4) is positioned at the upper back (between scapula) of individual 102. Sensing device 104(5) is positioned on an upper right arm of individual 102. Sensing device 104(6) is positioned on an upper left arm of individual 102. Sensing device 104(7) is positioned on a right forearm of individual 102. Sensing device 104(8) is positioned on a left forearm of individual 102. Sensing device 104(9) is positioned on the back of a right hand of individual 102. Sensing device 104(10) is positioned on the back of a left hand of individual 102. Sensing device 104(11) is positioned on a left thigh of individual 102. Sensing device 104(12) is positioned on a lower right leg of individual 102. Sensing device 104(13) is positioned on a lower left leg of individual 102. Sensing device 104(14) is positioned on a right foot of individual 102. Sensing device 104(15) is positioned on a left foot of individual 102.

More or fewer sensing devices 104, in the same or different body locations, may be configured with individual 102 without departing from the scope hereof. For example, to measure finger flexibility, gloves may be configured with a plurality of sensing devices 104 that measure movement of each finger segment. Sensing devices 104 may also be configured to measure other parameters, such as temperature, heart rate, etc., without departing from the scope hereof.

In one embodiment, each sensing device 104(1)-(15) measures motion (linear displacement and rotation) in three perpendicular axes X, Y and Z (often referred to as six axis measurement). Where individual 102 is to be monitored continuously (e.g., for an entire day or longer period), at least some of sensing devices 104 may be surgically implanted within individual 102. Certain other sensing devices 104 may be adhesively (e.g., as in a band aid) attached to individual 102. Certain other sensing devices 104 may be configured with clothing worn by individual 102. Sensing devices 104 may be configured with equipment (e.g., object handled by individual 102, exercise equipment, and so on). Sensing devices 104 may be selected to measure one or more of displacement, velocity, and acceleration. Alternatively these parameters may be derived from other inputs (e.g., video).

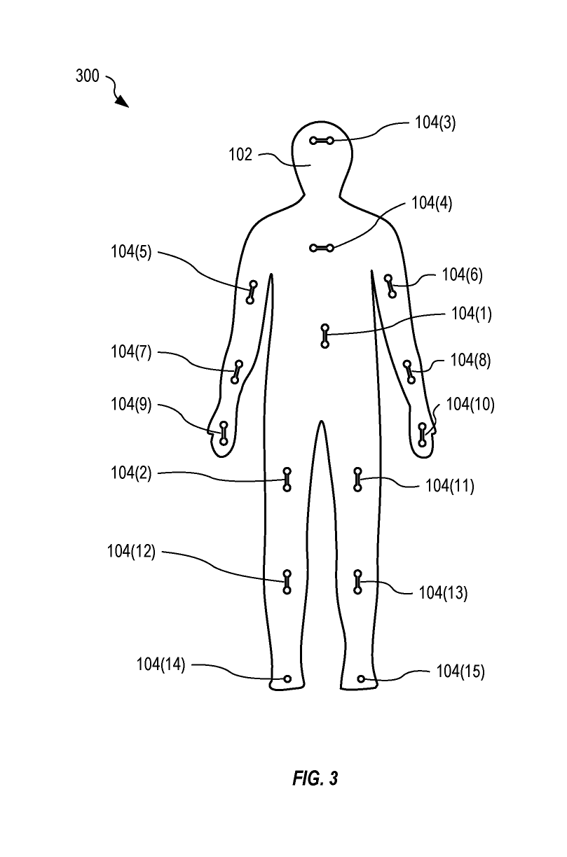

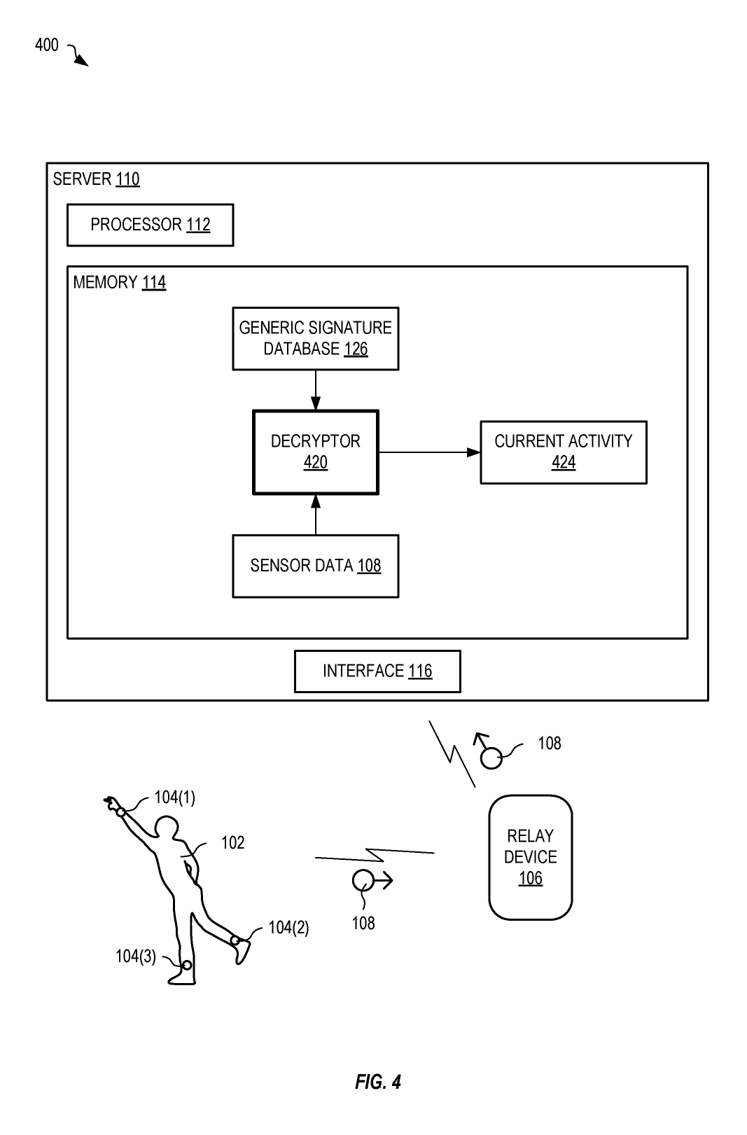

FIG. 4 shows one exemplary system 400 for capturing and decrypting sensor data 108 by using the generic signature database 126 of FIG. 1A. System 400 thereby determines the activity of individual 102 without any other input (e.g. visual). In the embodiment shown, system 400 is implemented within server 110 of FIG. 1A. System 400 includes a decryptor 420 that processes sensor data 108 against signatures within generic signature database 126 to determine a current activity 424 that defines the activity being performed by individual 102.

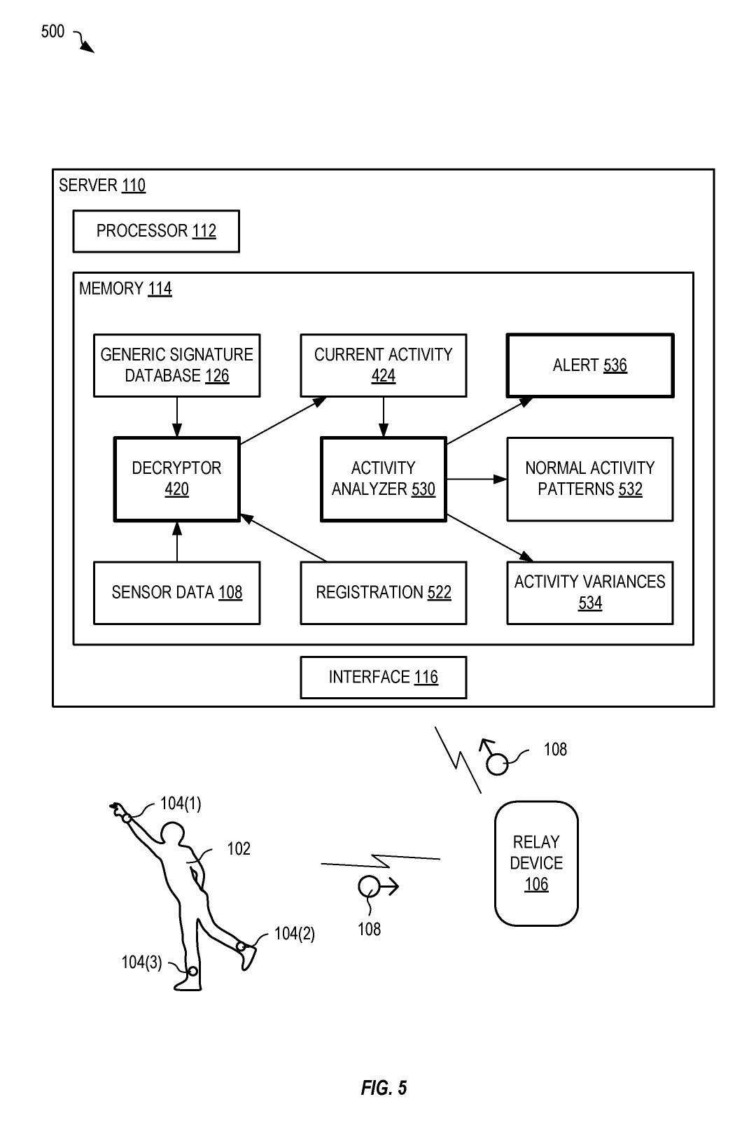

FIG. 5 shows one exemplary system 500 for detecting change in ability of an individual 102 based upon identified actions. In the embodiment shown, system 500 is implemented using server 110 of FIG. 1A.

Server 110 includes a registration database 522 that correlates individual 102 with each sensing device 104, and defines the type and location of each sensor on individual 102. For example, registration database 522 may define that sensing device 104(1) is positioned on a right wrist of individual 102, thereby indicating that movement information within sensor data 108 from sensing device 104(1) is indicative of movement of the wrist of individual 102.

Server 110 uses decryptor 420, implemented as machine readable instructions stored within memory 114 and executed by processor 112, to determine current activity 424 of individual 102. Decryptor 420 processes sensor data 108 against a generic signature database 126 and generates current activity 424 that defines the identified activities being performed by individual 102. Generic signature database 126 is for example a collection of sensor data signatures for one or more sensor types and positions that correspond to identified activities. Generic signature database 126 may represent “Big data” and may include signatures corresponding to identified activities of many individuals. Decryptor 420 thereby matches signatures derived from sensor data 108 to signatures defined within generic signature database 126 to determine current activity 424 of individual 102. Each activity may include one or more sub-activities, where each sub-activity is identified based upon at least one signature from a particular sensing device 104 configured with individual 102. In one example, current activity 424 includes a plurality of sub-activities that are each matched to sub-activities of a corresponding activity within generic signature database 126, where each sub-activity has one or core corresponding signatures that are matched to signatures of sensor data 108.

System 500 is a “learning system”, since generic signature database 126 may be updated as new activities are learned. For example, where sensor data 108 does not correlate to signatures within generic signature database 126, server 102 may send this sensor data 108 to an external device for a detailed review and/or a manual review, to identify the activity within sensor data 108. The identified activity may then be input to system 100, whereby generic signature database 126 is updated with the signatures to identify that activity. In one embodiment, generic signature database 126 is shared between a plurality of servers 110, each collecting and processing sensor data 108 from many individuals, whereby system 100 learns new activities very quickly.

Activities identified by decryptor 420 may include writing of the alphabet (in both cursive as well as print), opening of a jar, lifting and sipping of a coffee cup, cutting meat, buttoning a shirt, and so on. As described above, system 500 may learn any common conventional activity, such that it is defined within generic signature database 126 and used to determine current activity 424 based upon sensor data 108.

Server 110 also includes an activity analyzer 530 that processes current activity 424 to determine normal activity patterns 532 of individual 102. For example, where current activity 424 indicates that individual 102 is making a hot drink, and the current time is eight in the morning, activity analyzer 530 may determine that individual 102 frequently makes a hot drink at this time of the morning, and thereby define normal activity patterns 532 accordingly. More particularly, making the hot drink may include several identified sub-activities according to a specific pattern, such as reaching into a cupboard, spooning coffee into a coffee machine, spooning sugar into a cup, pouring coffee into the cup, and stirring the coffee and sugar in the cup. Thus, when activity analyzer 530 recognizes that current activity 424 matches an activity within normal activity patterns 532, activity analyzer 530 may predict, based upon normal activity patterns 532, a next activity of individual 102.

Over time, activity analyzer 530 collects and stores activities within normal activity patterns 532. This normal activity patterns 532 forms a history of activity of individual 102. Activity analyzer 530 further processes normal activity patterns 532 to determine activity variances 534 where more recent activity of individual 102 differs from previous normal activity patterns 532. For example, where individual 102 is injured, activity analyzer 530 may determines that when making coffee in the morning, individual 102 does not reach into the cupboard with the ease of previous activities.

In one embodiment, over a certain period, activity analyzer 530 processes current activity 424 and identifies handwriting of individual characters, wherein activity analyzer 530 may determine words, sentences, commends, and other input from individual 102 based upon these detected characters. System 500 may provide such input to other systems and devices.

In one embodiment, activity analyzer 530 generates an alert 536 indicating change in activity and/or ability of individual 102. In one embodiment, alert 536 is sent to individual 102, whereby individual 102 may comment on the change in activity behavior or ability. In another embodiment, alert 536 is sent to a care provider, where the care provider may follow-up with individual 102 regarding the change in activity and/or ability. In one embodiment, alert 536 is sent to other healthcare analytic systems for further analysis against healthcare data associated with individual 102 and others with similar conditions. For example, where activity analyzer 530 identifies a difference in an activity associated with making a hot beverage, activity analyzer 530 may generate alert 536 to indicate that individual 102 has become slower when making a hot beverage as compared to previous performances of this activity. In another example, where activity analyzer 530 identifies a difference in sub-activities associated with making the hot beverage, activity analyzer 530 may generate alert 536 to indicate that individual 102 has started omitting certain steps when making the hot beverage as compared to previous performances of this activity. Alert 536 may thus warn of dementia in individual 102. In another example, where activity analyzer 530 identifies that individual 102 has added one or more additional sub-activities when making the hot beverage, activity analyzer 530 may generate alert 536 to indicate that the activity has changed.

System 500 detects and analyzes activity of individual 102 based upon signature analysis of sensor data 108 within server 110. System 500 determines a current activity 424 of individual 102 based on analysis of motion information within sensor data 108. Utilizing this approach, system 500 determines change in actions performed by individual 102 over time and may thereby identify a malady or any other loss of ability that affects individual 102.

In another embodiment, individual 102 is a surgeon performing a standard operation, where in sensing devices 104 configured with individual 102 allow system 500 to monitor performance of individual 102 when performing that operation as compared to previous performances of that operation by individual 102 and/or other surgeons. For example, where a particular technique has increased success, system 500 may determine whether individual 102 is using and/or performing that technique.

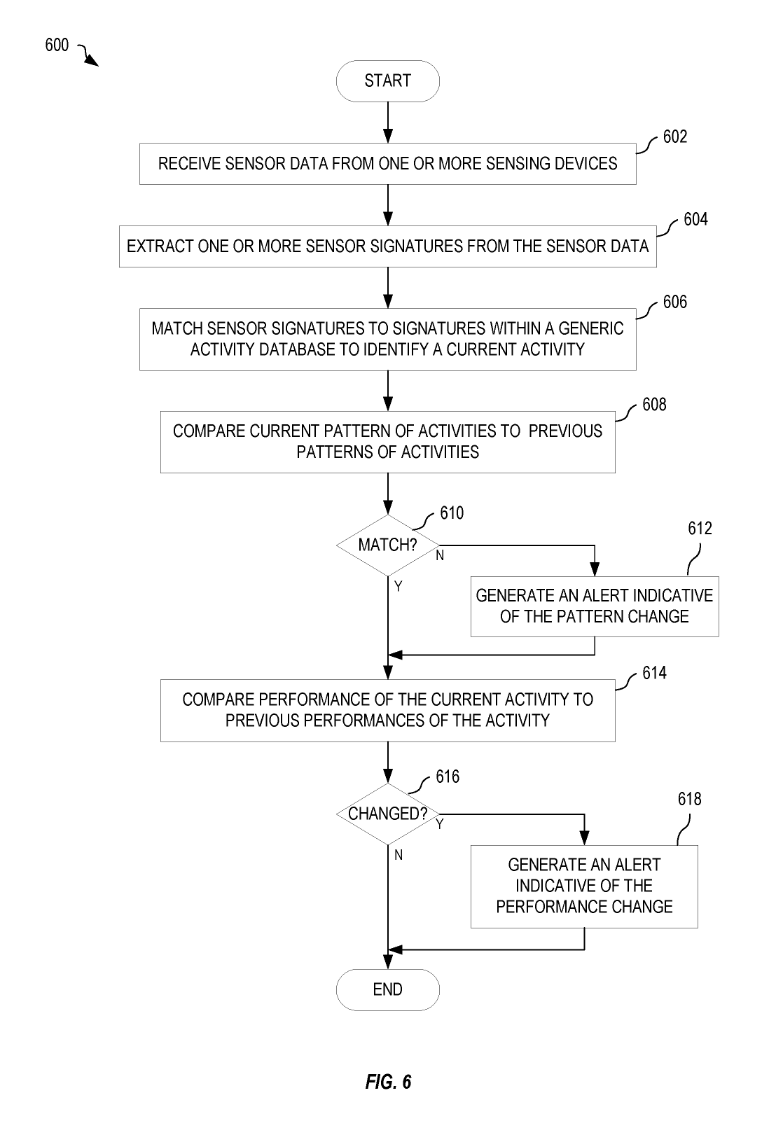

FIG. 6 is a flowchart illustrating one example method 600 for detecting change in ability of individual 102 based upon identified actions. Method 600 is implemented within decryptor 420 and activity analyzer 530 of server 110 of FIGS. 4 and 5, for example.

In step 602, method 600 receives sensor data from one or more sensing devices. In one example of step 602, decryptor 420 receives sensor data 108 from sensing devices 104, configured with individual 102, via relay device 106 and interface 116. In step 604, method 600 extracts one or more signatures from the sensor data. In one example of step 604, decryptor 420 extracts at least one signature from sensor data 108. In step 606, method 600 matches the sensor signatures of step 604 to signatures within a generic signature database to identify a corresponding activity. In one example of step 606, decryptor 420 matches the signature extracted from sensor data 108 to one or more signatures within generic signature database 126 to identify current activity 424.

In step 608, method 600 compares a current pattern of activities to previous patterns of activities. In one example of step 608, activity analyzer 530 compares sub-activities of current activity 424 against sub-activities of a corresponding activity within normal activity patterns 532.

Step 610 is a decision. If, in step 610, method 600 determines that the current pattern of activities does not match the previous or expected pattern of activities, method 600 continues with step 612; otherwise, method 600 continues with step 614. In step 612, method 600 generates an alert indicative of the pattern change. In one example of step 612, activity analyzer 530 generates alert 538 to indicate changes in the pattern of current activity 424 as compared to previous patterns within normal activity patterns 532. Method 600 then continues with step 614.

In step 614, method 600 compares performance of the current activity to previous performances of the activity. In one example of step 614, activity analyzer 530 compares performance of current activity 424 to previous performances of the same activity within normal activity patterns 532 to determine activity variances 534.

Step 616 is a decision. If, in step 616, method 600 determines that there are changes, method 600 continues with step 618; otherwise, method 600 terminates.

In step 618, method 600 generates an alert indicative of the change. In one example of step 618, activity analyzer 530 generates alert 538 to indicate changes in the performance of current activity 424 as compared to previous performances within normal activity patterns 532. Method 600 then terminates.

In the embodiments disclosed herein, on body or in body sensors capable of motion detection will be affixed. These sensors capture XY, YX and XZ motion in six degrees of freedom, time, circular and angular displacement torsion spherical coordinates or other motion and displacement parameters sensors may also capture velocity acceleration and other deformation, translation or translocation parameters.

Sensors may be of a wide range including one or more of strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, vibration sensors, and so on. Engineering has produced a plethora of sensors for all types of measurement and miniaturization of sensors and development of flexible circuitry allows movement sensors to be implanted within the body of individual or affixed on to the body of individual such that continuous flexibility measurement is possible without disruption of activity of individuals. See for example, www.MC10inc.com. e.g. the “Biostamp.” The embodiments hereof may use any type of sensor and any format as best suited to the measurements needed.

The motion, activity or activity component signature may be defined by the specific location of the sensors. The motion, activity or activity component signature may be defined by the specific resolution of the sensors. The motion, activity or activity component signature may be defined by the specific parameter measured by the sensing system—dimension/displacement, velocity, acceleration, angular rotation and the like.

The motion, activity or activity component signature may then be stored and a database created. The motion, activity or activity component signature may be further codified or encrypted. The motion, activity or activity component signature may be stored and refined over time from a continuously updated input stream (big data) and machine language smart system to “learn and improve” and otherwise enhance and refine the signature.

As more and more motions, activities or activity components are captured and codified or encrypted an effective code, lexicon and language of signatures will be established. As such, this language, code or lexicon may be utilized to decipher interpret, de-encrypt or otherwise translate a given motion signal or signature. In certain embodiments disclosed herein, this process may either be done manually, semi-manually or via an automated system.

In certain embodiments disclosed herein, the activity or activity component of an organism may be fully revealed, without visual or any other direct sensory input directly from the motion signature. For example, system 100 may be configured to allow input of sensor data 108, analysis and comparison to a known library and then via either manual or automated methods the activity will be revealed.

The captured and interpreted motion signature(s) may be analyzed (e.g., using system 600) over time or over differing circumstances or contexts. As such, alterations in the motion signature over time or context may reveal a decline in function, state of health or other endogenous or exogenous affecting condition being at work. Hence the motion signature may provide insight as to both the local and general status of function, health, mental or physical intactness or otherwise form and functionality of an individual.

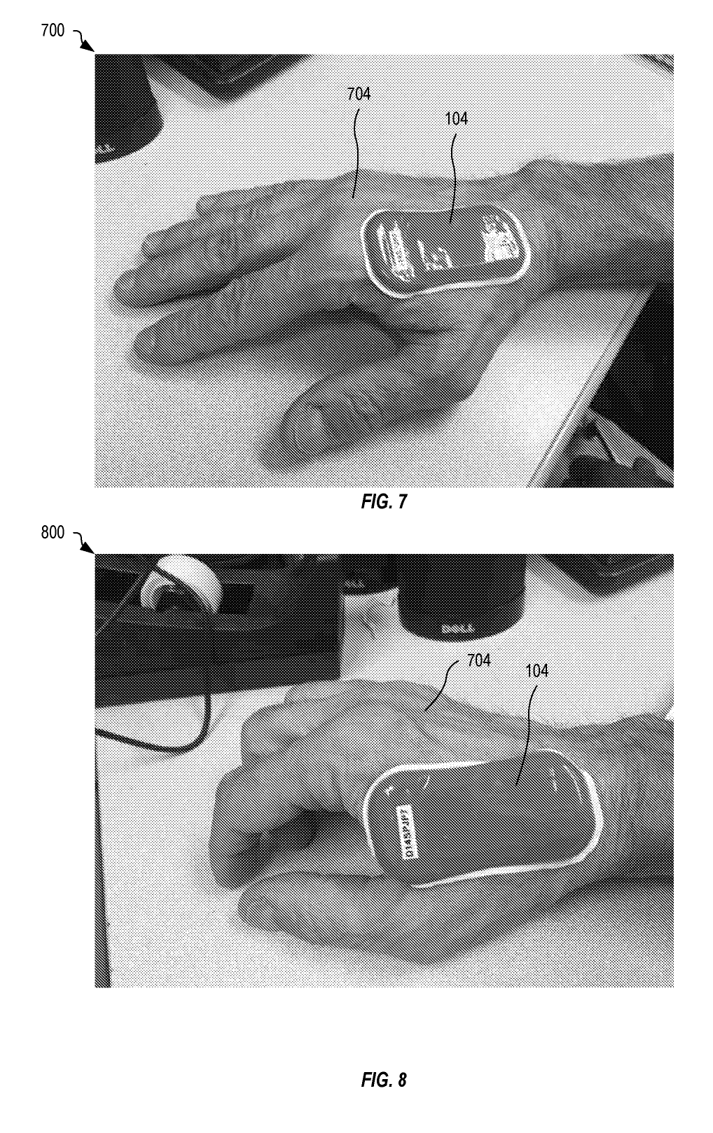

FIGS. 7 and 8 show one sensing device 104 (implemented as a “Biostamp” of www.MC10inc.com) attached to a hand 704 of individual 102. Using server 110 of FIG. 1, an intermittent stream of sensor data 108 is collected from sensing device 104. Decryptor 410 of system 400 processes this sensor data 108 to monitor, either automatic or a person (e.g., as a sentry), watch and act on sensor data 108 when a change occurs. As described above, where individual 102 is elderly and at home and where system 600 is remote therefrom, activity analyzer 530 determines, based upon the relayed sensor data 108, when individual 102 is not opening the refrigerator or drinking from a cup or opening a pill bottle as expected. System 600 may they generate alert 563, just on the basis of sensor data 108 and matched (or unmatched) signatures within generic signature database 126 and normal activity patterns 532, such that someone may be sent over to check on individual 102, make a call, contact via Skype or otherwise interact with individual 102 to correct a potential decline or adverse scenario that is evolving. System 600 may act as an either on line or otherwise early warning system of events that may be unfolding based on a change from the normal activity patterns of individual 102. Again, system 600 may detect both a delta of time as well a delta of quality or quantity of current activity 424.

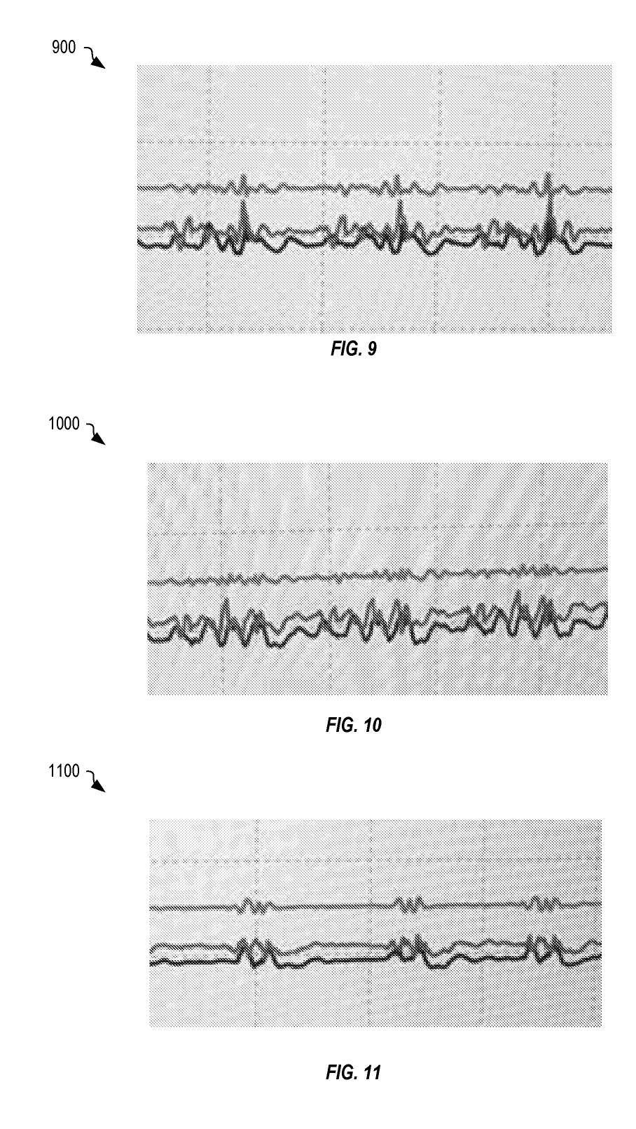

FIGS. 9, 10, and 11 shows graphs 900, 1000, and 1100 of exemplary sensor data 108 (i.e., collected from sensing device 104 of FIGS. 7 and 8, and individual 102 writes letters A, B and C, respectively, of the alphabet. Each graph shows data from multiple sensors of sensing device 104 as individual writes each letter three times.

Example motion signature capture scenarios include:
sensing devices positioned on an arm hand of individual 102 to detect motion of writing or opening a door or buttoning a shirt.
sensing devices positioned on leg(s) of individual 102 to detect when individual is sitting, standing, lying down, and/or arising from bed.
sensing devices positioned on a hand (w glove-like distribution) for opening a pill bottle.

In one embodiment, sensing device(s) 104 may be single, multiple, a cladding (e.g., like fish scales), on a glove, shirt, stocking, body suit.

As taught by FIG. 6 and associated description of Appendix A of U.S. Patent Application Ser. No. 62/351,264, sensing devices 104 may be configured within a room or area to capture motion of individual 102. Where these sensing devices are cameras can capture images, additional signal processing may occur to interpret these signatures and generate sensor data 108.

The embodiments described herein provide many advantages and technological features. For example, embodiments may capture motion signature for an activity and can collect, refine and create specific signature code or lexicon for a given activity or activity component such that the signature (whether dimensional—distance, velocity, acceleration or other physical parameter) is a surrogate defining equivalent i.e. a motion marker or a motion biomarker of that activity or act.

As such that activity may allow coding and encryption of an activity and then the ability to monitor over time and decode activity signals and then interpret the activity from the signature alone. Embodiments may interpret an activity and tell what it is from the signature alone without any other data (e.g., visual or other data). Embodiments may convert an activity related process into a code and then decode it.

Embodiments may encrypt and decrypt code, or may simply use digital signals. Embodiments may encode writing, activities of daily living—specifically claim, drinking from cup, glass, cutting food, opening pill bottle, buttoning shirt, closing a belt, zipping a zipper, putting on clothes, urinating, defecating, wiping body and cleaning after toilet use, rising from bed, washing face/body, brushing teeth, flossing, opening refrigerator, sitting in chair standing

Embodiments may utilize machine learning to continuously improve data base from big data population sources. Specific sensing devices (and sensors) may be used for capture of specific motion. Signatures may be defined for writing, handwriting penmanship and other script, print and inscribing means—of all languages may be encoded—e.g., Arabic characters, Chinese, Japanese, Korean and other language character forms. A digital pen or writing instrument may be used as a sensing device to allow a system for writing leading to codification. Software may be included to input code, interpret code and add warning to act. Embodiments may capture motion signatures with either on body or in body sensor or sensors. Sensor signals may be xy, yz, xz with 6 degrees of freedom, velocity, acceleration, angular motion. Embodiments may use all the sensor types including adherent stretchable electronic, Biostamp, flexible electronic and transient electronic systems, ultrasonic sensors, radiofrequency, radio transmitter, etc. Sensor forms may be single, multiple, multiplexed, clad, gloves, shirt, stocking, adherent, and conformal. Capture, analysis and telemetry may utilize the “cloud” and data may be retrieved from the cloud. These advantages provide efficient and cost effective systems and methods.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. In particular, the following embodiments are specifically contemplated, as well as any combinations of such embodiments that are compatible with one another:

(A) A system for detecting changes in ability of an individual to perform an activity. The system includes a processor, a memory, a generic signature database stored within the memory and having at least one signature that corresponds to the activity, a decryptor that identifies a current activity being performed by the individual by matching one or more signatures, determined from sensor data received from sensing devices attached to the individual, to the at least one signature within the generic signature database, a normal activity pattern database stored within the memory and that includes a pattern of the activity previously determined for the individual, and an activity analyzer configured to compare performance of the current activity to performance of the previous activity to identify change in the performance, and to generate an alert indicating the change when the change is identified.

(B) In the system denoted as (A), the one or more signatures determined from the sensor data having one or more of displacement, velocity, acceleration, rotation.

(C) In either of the systems denoted as (A) and (B), the decryptor being further configured to determine, from one or more of distance, velocity, and acceleration within the sensor data, a motion signature for the current activity and to collect, refine and create specific signature codes for a given activity or activity component, wherein the motion signature is a surrogate defining equivalent such as a motion marker or a motion biomarker of a that activity or act.

(D) In any of the systems denoted as (A)-(C), the decryptor being further configured to allow coding and encryption of the sensor data and to monitor over time and decode subsequent sensor data and to interpret the activity from the signature alone.

(E) In any of the systems denoted as (A)-(D), the decryptor being further configured to interpret the sensor data and determine the current activity based upon the signature alone and without any other visual or other data.

(F) In any of the systems denoted as (A)-(E), the decryptor being further configured to convert the sensor data for an activity related process into a code and to decode the code to determine the activity.

(G) In any of the systems denoted as (A)-(F), the code being encrypted and decrypted to determine the activity.

(H) In any of the systems denoted as (A)-(G), the code corresponding to activities of daily living, including one or more of writing, drinking from cup, drinking from a glass, cutting food, opening a pill bottle, buttoning a shirt, closing a belt, zipping a zipper, putting on clothes, urinating, defecating, wiping body and cleaning after toilet use, rising from bed, washing face/body, brushing teeth, flossing, opening refrigerator, and sitting in chair and then standing.

(I) In any of the systems denoted as (A)-(H), the decryptor and the activity analyzer being further configured for machine learning to continuously improving the generic signature database and the normal activity pattern database based upon big data population sources.

(J) In any of the systems denoted as (A)-(I), the sensing devices including one or more sensors selected from the group including strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, and vibration sensors.

(K) In any of the systems denoted as (A)-(J), the current activity being selected from the group including writing, handwriting penmanship and other script, print and inscribing means of all languages including Arabic characters, Chinese, Japanese, Korean and other language character forms.

(L) In any of the systems denoted as (A)-(K), the sensing devices further comprising a digital pen or writing instrument for codification of writing.

(M) In any of the systems denoted as (A)-(L), one or more of the sensors of the sensing devices being configured on a body of the individual.

(N) In any of the systems denoted as (A)-(M), one or more of the sensors of the sensing devices being configured in the body of the individual.

(O) In any of the systems denoted as (A)-(N), one or more of the sensors of the sensing devices being configured to sense six degrees of freedom for one or more of velocity, acceleration, and angular motion.

(P) In any of the systems denoted as (A)-(O), one or more of the sensors of the sensing devices having a sensor type selected from the group including adherent stretchable electronic, Biostamp, flexible electronic and transient electronic systems, ultrasonic sensors, radiofrequency, and radio transmitter.

(Q) In any of the systems denoted as (A)-(P), one or more of the sensors of the sensing devices having a sensor form selected from the group including single, multiple, multiplexed, clad, gloves, shirt, stocking, adherent, and conformal.

(R) In any of the systems denoted as (A)-(Q), one or more of the sensing devices being capable of capture, analysis and telemetry of data to the cloud and data retrieval from the cloud.

(S) In any of the systems denoted as (A)-(R), the functionality being implemented by software to input code, to interpret code, and to add warning to act.

(T) A method for detecting changes in ability of an individual to perform an activity. Sensor data indicative of movement of the individual is processed to identify a current activity of the individual. Performance of the current activity is compared to previous performances of the activity by the individual to identify change in the ability of the individual to perform the activity. An alert is generated to indicate the change in the ability of the individual to perform the activity.

(U) The method denoted as (T), further including receiving the sensor data from at least one sensing device configured with the individual.

(V) In either of the methods denoted as (T) and (U), the sensor data having at least one of displacement, movement, velocity, and acceleration of the individual as measured by at least one sensor of the at least one sensing device.

(W) In any of the methods denoted as (T)-(V), the step of processing including determining a signature from the sensor data and matching the signature to one of a plurality of signatures within a generic signature database that corresponds to the identified activity.

What is claimed is:

1. A system for detecting changes in ability of an individual to perform an activity, comprising:
a processor;
a memory;
a generic signature database stored within the memory and comprising at least one signature that corresponds to the activity;
a decryptor that identifies a current activity being performed by the individual by matching one or more signatures, determined from sensor data received from sensing devices attached to the individual, to the at least one signature within the generic signature database;
a normal activity pattern database stored within the memory and that comprises a pattern of the activity previously determined for the individual; and
an activity analyzer configured to compare performance of the current activity to performance of the previous activity to identify change in the performance, and to generate an alert indicating the change when the change is identified.

2. The system of claim 1, the one or more signatures determined from the sensor data comprising one or more of displacement, velocity, acceleration, rotation.

3. The system of claim 2, the decryptor further configured to determine, from one or more of distance, velocity, and acceleration within the sensor data, a motion signature for the current activity and to collect, refine and create specific signature codes for a given activity or activity component, wherein the motion signature is a surrogate defining equivalent such as a motion marker or a motion biomarker of a that activity or act.

4. The system of claim 3, the decryptor further configured to allow coding and encryption of the sensor data and to monitor over time and decode subsequent sensor data and to interpret the activity from the signature alone.

5. The system of claim 4, the decryptor further configured to interpret the sensor data and determine the current activity based upon the signature alone and without any other visual or other data.

6. The system of claim 5, the decryptor further configured to convert the sensor data for an activity related process into a code and to decode the code to determine the activity.

7. The system of claim 6, wherein the code is encrypted and decrypted to determine the activity.

8. The system of claim 7, wherein the code corresponds to activities of daily living, including one or more of writing, drinking from cup, drinking from a glass, cutting food, opening a pill bottle, buttoning a shirt, closing a belt, zipping a zipper, putting on clothes, urinating, defecating, wiping body and cleaning after toilet use, rising from bed, washing face/body, brushing teeth, flossing, opening refrigerator, and sitting in chair and then standing.

9. The system of claim 8, the decryptor and the activity analyzer further configured for machine learning to continuously improving the generic signature database and the normal activity pattern database based upon big data population sources.

10. The system of claim 9, the sensing devices comprising one or more sensors selected from the group including strain gauges, accelerometers, gyroscopes, displacement sensors, proximity sensors, hall effect sensors, optical encoders, potentiometers, linear and rotary sensors, eddy-current sensors, reflective light sensors, pressure sensors, force sensors, tilt sensors, and vibration sensors.

11. The system of claim 10, the current activity selected from the group including writing, handwriting penmanship and other script, print and inscribing means of all languages including Arabic characters, Chinese, Japanese, Korean and other language character forms.

12. The system of claim 11, the sensing devices further comprising a digital pen or writing instrument for codification of writing.

13. The system of claim 12, wherein one or more of the sensors of the sensing devices being configured on a body of the individual.

14. The system of claim 13, wherein one or more of the sensors of the sensing devices being configured in the body of the individual.

15. The system of claim 14, wherein one or more of the sensors of the sensing devices being configured to sense six degrees of freedom for one or more of velocity, acceleration, and angular motion.

16. The system of claim 15, wherein one or more of the sensors of the sensing devices have a sensor type selected from the group including adherent stretchable electronic, Biostamp, flexible electronic and transient electronic systems, ultrasonic sensors, radiofrequency, and radio transmitter.

17. The system of claim 16, wherein one or more of the sensors of the sensing devices have a sensor form selected from the group including single, multiple, multiplexed, clad, gloves, shirt, stocking, adherent, and conformal.

18. The system of claim 17, wherein one or more of the sensing devices are capable of capture, analysis and telemetry of data to the cloud and data retrieval from the cloud.

19. The system of claim 18, wherein the functionality is implemented by software to input code, to interpret code, and to add warning to act.

20. A method for detecting changes in ability of an individual to perform an activity, comprising:
processing sensor data indicative of movement of the individual to identify a current activity of the individual;
comparing performance of the current activity to previous performances of the activity by the individual to identify change in the ability of the individual to perform the activity; and
generating an alert to indicate the change in the ability of the individual to perform the activity.

21. The method of claim 20, further comprising receiving the sensor data from at least one sensing device configured with the individual.

22. The method of claim 21, the sensor data comprising at least one of displacement, movement, velocity, and acceleration of the individual as measured by at least one sensor of the at least one sensing device.

23. The method of claim 22, the step of processing comprising determining a signature from the sensor data and matching the signature to one of a plurality of signatures within a generic signature database that corresponds to the identified activity.
